(19) United States
(12) Patent Application Publication
(10) Pub. No.: US 2024/0042713 A1
(43) Pub. Date: Feb. 8, 2024

(54) EXTRUDED OCULAR INSERTS OR IMPLANTS AND METHODS THEREOF

(71) Applicant: OCULAR THERAPEUTIX, INC., Bedford, MA (US)

(72) Inventors: Peter JARRETT, Burlington, MA (US); Rami EL-HAYEK, Norwood, MA (US); Erica KAHN, Cambridge, MA (US); Dinesh HASWANI, Acton, MA (US); Eric DICKINSON, Amherst, NH (US); Timothy S. JARRETT, Boston, MA (US); Michael MCGRATH, Bedford, MA (US)

(21) Appl. No.: 18/273,145

(22) PCT Filed: Dec. 6, 2022
(86) PCT No.: PCT/US2022/051993

§ 371 Date: Jul. 19, 2023

Related U.S. Application Data
(60) Provisional application No. 63/286,515, filed on Dec. 6, 2021.
(60) Provisional application No. 63/300,434, filed on Jan. 18, 2022.

(51) Int. Cl. B29D 11/00; A61K 31/573; A61K 9/00

(52) U.S. Cl. CPC B29D 11/0074; A61K 31/573; A61K 9/0051; B29K 2071/02

(57) ABSTRACT

The present invention is directed to a method of preparing a sustained release biodegradable ocular insert or implant comprising melt extruding or injection molding a polymer composition and an active agent to form an insert or implant suitable for administration to the body. e.g., ocular administration. The method comprises feeding the polymer composition and the active into an extruder; mixing the components in the extruder; extruding a strand; and cutting the strand into unit dose inserts or implants.

### TECHNICAL FIELD

The present invention relates to extruded ocular inserts or implants. The inserts or implants can be suitable for insertion or implantation in the human body, e.g., subcutaneous, subconjunctival, intracanalicular, intracameral, suprachoroidal, fornix or intravitreal administration for various diseases and disorders, including diseases and disorders of the eye.

### BACKGROUND

Ocular inserts or implants are important therapeutic options as they can provide prolonged treatment without the need for constant and repeated administration with drops.

Small scale manufacturing processes for hydrogel ocular inserts or implants include a casting process which involves forming a reacting mixture of hydrogel precursor solutions with suspended drug particles and injection molding the hydrogel inside a tubular mold.

For large scale manufacturing processes, the casting process has scale-up and efficiency challenges due to limitations of the manufacturing equipment.

There exists a need in the art for ocular inserts or implants and processes that can be efficiently scaled to accomodate large scale manufacturing processes.

### OBJECTS AND SUMMARY OF THE INVENTION

It is an object of certain embodiments of the present invention to provide an extruded ocular insert or implant.

Another object of certain embodiments of the present invention is to provide methods of preparing extruded ocular inserts or implants.

Another object of certain embodiments of the present invention is to provide methods of treating diseases and conditions of the eye comprising administering an extruded ocular insert or implant as disclosed herein.

One or more objects of the invention may be met by the present invention which in certain embodiments is directed to a method of preparing a sustained release biodegradable ocular insert or implant comprising extruding a polymer composition and an active agent to form an insert or implant suitable for ocular administration.

In certain embodiments, the present invention is directed to an ocular insert or implant prepared by a method as disclosed herein.

In certain embodiments, the present invention is directed to a method of treating an ocular disease comprising administering an ocular insert or implant as disclosed herein.

One or more of these objects of the present invention and others are solved by one or more embodiments of the invention as disclosed and claimed herein.

### BRIEF DESCRIPTION OF THE DRAWINGS

FIG. 1 depicts in vitro release of melt extruded material.

FIG. 2 shows an exemplary custom parallel twin screw configuration as disclosed in the Examples.

### DEFINITIONS

The term “insert” as used herein refers to an object that contains an active agent, e.g., a glucocorticoid such as dexamethasone and that is administered into the human or animal body via an existing opening, such as to the canaliculus of the eye, where it remains for a certain period of time while it releases the active agent into the surrounding environment. The term “implant” as used herein refers to an object that contains an active agent, e.g., a glucocorticoid such as dexamethasone and that is administered into the human or animal body via injection or surgical implantation, such as to the vitreous humor of the eye, where it remains for a certain period of time while it releases the active agent into the surrounding environment. An insert or implant has a predetermined shape before being inserted or implanted, which general shape is maintained to a certain degree upon placing the insert or implant into the desired location, although dimensions of the insert or implant (e.g. length and/or diameter) may change after administration due to hydration as further disclosed herein. In other words, what is administered into the body is not a solution or suspension, but an already shaped, coherent object. The insert or implant has thus been completely formed as disclosed herein prior to being administered.

Over the course of time the insert or implant may biodegrade (as disclosed herein), may thereby change its shape (e.g. may expand in diameter and decrease in length) until it has been completely dissolved/resorbed. Herein, the terms “insert” of “implant” are used to refer both to an insert or implant in a hydrated (also referred to herein as “wet”) state when it contains water, e.g. after the insert or implant has been (re-)hydrated once administered to the body, e.g. the eye, or otherwise immersed into an aqueous environment, and to an insert or implant in its/a dry (dried/dehydrated) state.

The term “ocular” as used herein refers to the eye in general, or any part or portion of the eye (as an “ocular insert” or “ocular implant” according to the invention refers to an insert or implant that can in principle be administered to any part or portion of the eye). The present invention in certain embodiments is directed to intracanalicular administration of an ocular insert, and to the treatment of, e.g., dry eye disease (DED) or pain after surgery, as further disclosed herein.

The term “biodegradable” as used herein refers to a material or object (such as the intracanalicular insert or implant according to the present invention) which becomes degraded in vivo, i.e., when placed in the human or animal body. In the context of the present invention, as disclosed in detail herein, the insert or implant comprising the hydrogel within which particles of an active agent are dispersed, slowly biodegrades over time once deposited within the body or eye, e.g., within the canaliculus. In certain embodiments, biodegradation takes place at least in part via ester hydrolysis in the aqueous environment provided by the tear fluid. In certain embodiments, the intracanalicular inserts or implants of the present invention slowly soften and liquefy, and are eventually cleared (disposed/washed out) through the nasolacrimal duct.

A “hydrogel” is a three-dimensional network of hydrophilic natural or synthetic polymers (as disclosed herein) that can swell in water and hold an amount of water (e.g., greater than 25%, greater than 50%, greater than 75% or from 25% to about 90% or from about 705 to about 99%) while maintaining or substantially maintaining its structure, e.g., due to chemical or physical cross-linking of individual polymer chains. Due to their high water content, hydrogels are soft and flexible, which makes them very similar to natural tissue. In the present invention the term “hydrogel” is used to refer both to a hydrogel in the hydrated state when it contains water (e.g. after the hydrogel has been formed in an aqueous solution, or after the hydrogel has been (re-) hydrated once inserted or implanted into the eye or otherwise immersed into an aqueous environment) and to a hydrogel in its/a dry (dried/dehydrated) state, also called a xerogel, when it has been dried to a low water content of e.g. not more than 1% by weight. In the present invention, wherein an active principle is contained (e.g. dispersed) in a hydrogel, the hydrogel may also be referred to as a “matrix”.

The term “polymer network” as used herein describes a structure formed of polymer chains (of the same or different molecular structure and of the same or different average molecular weight) that are cross-linked with each other. Types of polymers suitable for the purposes of the present invention are disclosed herein. The polymer network may be formed with the aid of a crosslinking agent as also disclosed herein.

The term “amorphous” refers to a polymer or polymer network which does not exhibit crystalline structures in X-ray or electron scattering experiments.

The term “semi-crystalline” refers to a polymer or polymer network which possesses some crystalline character, i.e., exhibits some crystalline properties in X-ray or electron scattering experiments.

The term “precursor” or “polymer precursor” herein refers to those molecules or compounds that are reacted with each other and that, once reacted, are thus connected via crosslinks to form the polymer network and thus the hydrogel matrix. While other materials might be present in the hydrogel, such as active agents, visualization agents or buffers, they are not referred to as “precursors”.

The molecular weight of a polymer precursor as used for the purposes of the present invention and as disclosed herein may be determined by analytical methods known in the art. The molecular weight of polyethylene glycol can for example be determined by any method known in the art, including gel electrophoresis such as SDS-PAGE (sodium dodecyl sulphate-polyacrylamide gel electrophoresis), gel permeation chromatography (GPC), including GPC with dynamic light scattering (DLS), liquid chromatography (LC), as well as mass spectrometry such as matrix-assisted laser desorption/ionization-time of flight (MALDI-TOF) spectrometry or electrospray ionization (ESI) mass spectrometry. The molecular weight of a polymer, including a polyethylene glycol precursor as disclosed herein, is an average molecular weight (based on the polymer's molecular weight distribution), and may therefore be indicated by means of various average values, including the weight average molecular weight (Mw) and the number average molecular weight (Mn). Any of such average values may be used in the context of the present invention. In certain embodiments, the average molecular weight of the polyethylene glycol units or other precursors as disclosed herein is the number average molecular weight.

The parts of the precursor molecules that are still present in the final polymer network are also called “units” herein. The “units” are thus the building blocks or constituents of the polymer network forming the hydrogel. For example, a polymer network suitable for use in the present invention may contain identical or different polyethylene glycol units as further disclosed herein.

As used herein, the term “crosslinking agent” refers to any molecule that is suitable for connecting precursors via crosslinks to form the polymer network and thus the hydrogel matrix. Crosslinking agents may be low-molecular weight compounds or may be polymeric compounds as disclosed herein.

The term “sustained release” is defined for the purposes of the present invention to refer to pharmaceutical dosage forms which are formulated to make an active agent available over an extended period of time after administration, such as one or more weeks, thereby allowing a reduction in dosing frequency compared to an immediate release dosage form, e.g. a solution of an active agent that is topically applied onto the eye (e.g., glucocorticoid-comprising eye drops). Other terms that may be used herein interchangeably with “sustained release” are “extended release” or “controlled release”. Within the meaning of the invention, the term “sustained release” also comprises a period of constant active agent release per day, which may be followed by a period of tapered active agent release. In other words, during a “sustained release” period, the release rate is not necessarily constant or essentially constant, but may change over time. Within the meaning of the invention, the term “tapered” or “tapering” refers to a decreasing rate of release of active agent such as dexamethasone over time, e.g., until the active agent is completely released.

The term “visualization agent” as used herein refers to a molecule or moiety that may be contained within an insert or implant of the present invention and that provides the possibility of easily visualizing the insert or implant in a non-invasive manner when it is located in the body, e.g., the canaliculus of the eye, e.g. by illuminating the corresponding eye parts with a suitable light source.

As used herein, the term “ocular surface” comprises the conjunctiva and the cornea, together with elements such as the lacrimal apparatus, including the lacrimal punctum, as well as the lacrimal canaliculus and associated eyelid structures. Within the meaning of this invention, the ocular surface encompasses also the aqueous humor.

As used herein, the terms “tear fluid” or “tears” or “tear film” refer to the clear liquid secreted by the lacrimal glands, which lubricates the eyes. Tears are made up of water, electrolytes, proteins, lipids, and mucins.

As used herein, the term “bilaterally” or “bilateral” refers (in the context of administration of the inserts or implants of the present invention) to an administration of the inserts or implants into both eyes of a patient. “Unilaterally” or “unilateral” thus refers to an administration of the insert or implant into one eye only. The inserts or implants may be inserted into the superior and/or the inferior canaliculus of both eyes or of one eye.

As used herein, the terms “administration” or “administering” or “administered” etc. in the context of the sustained release biodegradable inserts or implants of the present invention refer to the process of insertion, injection or surgical implantation of the inserts or implants into the body or eye. In certain embodiments, the formulation is inserted through the opening of the punctum into the canaliculus of the eye. The terms “administration” or “administering” or “administered” etc. in the context of topical ophthalmic pharmacological products such as eye drops (which are not the subject of the present invention) refer to topical application of these products onto the eye.

As used herein, the term “insert stacking” or “stacking” refers to the insertion of a further insert or implant on top of a first insert or implant (e.g., intracanalicularly) while the first insert or implant is still retained. In certain embodiments, the further insert or implant is placed on top of the first insert or implant after the active agent contained in the first insert or implant is essentially completely released, or after at least about 70% or at least about 80% or at least about 90% of the active contained in the first insert or implant has been released. Insert or implant stacking enables, for instance, prolonged active agent treatment.

The term “plug” as used herein refers to a device capable of providing an occlusion of the tear duct(s) (“lacrimal occlusion”) thereby preventing draining of tears. A plug thus increases tear retention, which helps to keep the eyes moist. Plugs can be classified into “punctal plugs” and “intracanalicular plugs”. Intracanalicular plugs are also referred to as “canalicular plugs” in literature. Both plug classes are inserted through the upper and/or lower punctum of the eye.

Punctal plugs rest at the punctal opening making them easily visible and, hence, removable without much difficulty. However, punctal plugs may show poor retention rates and can be more easily contaminated with microbes due to their exposed localization resulting in infection. In contrast, intracanalicular plugs are essentially not visible and provide a better retention rate compared to punctal plugs as they are placed inside either the vertical or the horizontal canaliculus. However, currently available intracanalicular plugs may not be easy to remove and/or may provide an increased risk of migration due to loose fit. Commercially available plugs are often made of collagen, acrylic polymers, or silicone.

The terms “canaliculus” (plural “canaliculi”) or alternatively “tear duct” as used herein refer to the lacrimal canaliculus, i.e. the small channels in each eyelid that drain lacrimal fluid (tear fluid) from the lacrimal punctum to the nasolacrimal duct. Canaliculi therefore form part of the lacrimal apparatus that drains lacrimal fluid from the ocular surface to the nasal cavity. The canaliculus in the upper eyelid is referred to as “superior canaliculus” or “upper canaliculus”, whereas the canaliculus in the lower eyelid is referred to as “inferior canaliculus” or “lower canaliculus”. Each canaliculus comprises a vertical region, referred to as “vertical canaliculus” following the lacrimal punctum and a horizontal region, referred to as “horizontal canaliculus” following the vertical canaliculus, wherein the horizontal canaliculus merges into the nasolacrimal duct.

The term “punctum” (plural “puncta”) refers to the lacrimal punctum, an opening on the margins of the eyelids, representing the entrance to the canaliculus. After tears are produced, some fluid evaporates between blinks, and some is drained through the lacrimal punctum. As both the upper and the lower eyelids show the lacrimal punctum, the puncta are therefore referred to as “upper punctum” or “superior punctum” and “lower punctum” or “inferior punctum”.

The term “intracanalicular insert” refers to an insert that can be administered through the upper and/or lower punctum into the superior and/or inferior canaliculus of the eye, in particular into the superior and/or inferior vertical canaliculus of the eye. Due to the intracanalicular localization of the insert, the insert blocks tear drainage through lacrimal occlusion such as also observed for intracanalicular plugs. The intracanalicular inserts of the present invention may be insert inserted bilaterally or unilaterally into the inferior and/or superior vertical canaliculi of the eyes. According to the present invention, the intracanalicular insert is a sustained release biodegradable insert.

The terms “API”, “active (pharmaceutical) ingredient”, “active (pharmaceutical) agent”, “active (pharmaceutical) principle”, “(active) therapeutic agent”, “active”, and “drug” are used interchangeably herein and refer to the substance used in a finished pharmaceutical product (FPP) as well as the substance used in the preparation of such a finished pharmaceutical product, intended to furnish pharmacological activity or to otherwise have direct effect in the diagnosis, cure, mitigation, treatment or prevention of a disease, or to have direct effect in restoring, correcting or modifying physiological functions in a patient.

For the purposes of the present invention, active agents in all their possible forms, including any active agent polymorphs or any pharmaceutically acceptable salts, anhydrates, hydrates, other solvates or derivatives of active agents, can be used. Whenever in this description or in the claims an active agent is referred to by name, e.g., “dexamethasone”, even if not explicitly stated, it also refers to any such pharmaceutically acceptable polymorphs, salts, anhydrates, solvates (including hydrates) or derivatives of the active agent.

As used herein, the term “therapeutically effective” refers to the amount of drug or active agent (e.g., glucocorticoid) required to produce a desired therapeutic response or result after administration.

The term “average” as used herein refers to a central or typical value in a set of data, which is calculated by dividing the sum of the values in the set by their number.

As used herein, the term “about” in connection with a measured quantity refers to the normal variations in that measured quantity, as expected by one of ordinary skill in the art in making the measurement and exercising a level of care commensurate with the objective of measurement and the precision of the measuring equipment.

As used herein, the term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that.

As used herein, the singular forms “a,” “an”, and “the” include plural references unless the context clearly indicates otherwise.

The term “and/or” as used in a phrase such as “A and/or B” herein is intended to include both “A and B” and “A or B”.

Open terms such as “include,” “including,” “contain,” “containing” and the like as used herein mean “comprising” and are intended to refer to open-ended lists or enumerations of elements, method steps, or the like and are thus not intended to be limited to the recited elements, method steps or the like but are intended to also include additional, unrecited elements, method steps or the like.

The term “up to” when used herein together with a certain value or number is meant to include the respective value or number. For example, the term “up to 25 days” means “up to and including 25 days”.

All references disclosed herein are hereby incorporated by reference in their entireties for all purposes (with the instant specification prevailing in case of conflict).

### DETAILED DESCRIPTION

In certain embodiments the present invention is directed to a method of preparing a sustained release biodegradable ocular insert or implant comprising melt extruding or injection molding a polymer composition and an active agent to form an insert or implant suitable for administration to the body, e.g., ocular administration.

In other embodiments, the method comprises feeding the polymer composition and the active agent into an extruder; mixing the components in the extruder; extruding a strand; and cutting the strand into unit dose inserts or implants.

In certain embodiments, the polymer composition and the active agent are fed separately into the extruder. In other embodiments, the polymer composition and active agent are fed simultaneously into the extruder. In certain embodiments, the polymer composition are pre-mixed, e.g., melt blended, prior to introduction into the extruder. The mixing can be by a method using, e.g., an orbital mixer, an acoustic mixer or a v-shell blender. In certain embodiments, the polymer composition and active agent are melt blended, milled and then fed into the extruder.

In certain embodiments, the method further comprising cooling the strand, e.g., prior to cutting the strand.

In certain embodiments, the method further comprises stretching the strand, e.g., prior to cutting the strand.

In certain embodiments, the stretching is performed under wet or humid conditions, heated conditions, or a combination thereof. In other embodiments, the stretching is performed under dry conditions, heated conditions, or a combination thereof. In certain embodiments, strands that are stretched after crosslinking in a high humidity environment, e.g., a humidity chamber, may have shape memory or partial shape memory when placed in an aqueous environment after drying. In certain embodiments, strands that are stretched or otherwise made to have smaller diameters immediately after extrusion and before crosslinking when still warm may not have shape memory.

In certain embodiments, the extruded composition is subject to a curing step, e.g., humidity exposure. When one reactant is a salt, e.g., a salt of an amine, the salt is insoluble in the dry polymer melt. In this case, curing is accomplished by exposing the dry, extruded composition to humidity and allowing the extruded composition to imbibe water from the surroundings, thus allowing the salt to solubilize and react to crosslink the precursors and form a matrix. In certain embodiments, the curing crosslinks the polymer composition.

In certain embodiments, the method further comprises drying the strand after stretching the strand.

In other embodiments, any of the method steps disclosed herein can be performed simultaneously or sequentially in any order.

In certain embodiments, the method further comprises melting the polymer in the extruder at a temperature below the melting point of the active agent. The optimal temperature of the molten polymer is determined experimentally by its extrusion properties. In certain embodiments, the unmelted active agent remains unchanged through this melt extrusion process. In certain embodiments, the extrusion is performed above the melting point of the polymer and the active agent. This may result in a color change and/or change in form of the active agent, e.g., from amorphous to crystalline. The temperature can be, e.g., less than about 180°, less than about 150°, less than about 130°, less than about 120°, less than about 100°, less than about 90°, less than about 80°, less than about 70°, less than about 60°, less than about 50°. In some embodiments, the temperature is from about 50° to about 80° C. In other embodiments, the temperature is from about 50° to about 200°, about 60° to about 180° or about 80° to about 140°. An exlepary temperature is about 40° to about 90°. By virtue of certain embodiments of the present invention, the temperature is keep as low as possible to protect excipient powders and active ingredient and to optimize stability. In certain embodiments, the active agent is axitinib and the polymer is melted in the extruder at a temperature from 57° C. to about 200° C., from about to about 150° C. or from about 70° C. to about 90° C. In certain embodiments, the the active agent is dexamethasone and the polymer is melted in the extruder at a temperature from 57° C. to about 250° C., from about 65° C. to about 175° C. or from about 70° C. to about 90° C. In certain embodiments, the active agent is cyclosporine and the polymer is melted in the extruder at a temperature from 57° C. to about 145° C., from about 65° C. to about 120° C. or from about 70° C. to about 90° C. In certain embodiments, the active agent is bupivacaine and the polymer is melted in the extruder at a temperature from 57° C. to about 105° C., from about 65° C. to about 95° C. or from about 70° C. to about 90° C.

In certain embodiments, the extruded composition is dried, when in strand form or in unit doses. In certain embodiments, the drying is performed after stretching the strand. The drying can be, e.g., evaporative drying at ambient temperatures or can include heat, vacuum or a combination thereof.

In certain embodiments, the hydrogel strand is stretched by a stretch factor in the range of about 1.1 to about 10, 1.2 to about 6 or about 1.5 to about 4.

In certain embodiments, the strand is cut into segments having an average length of equal to or less than about 20 mm, 17 mm, 15 mm, 12 mm, 10 mm, 8 mm, 5 mm, 4 mm, 3 mm, 2 mm, 1 mm or 0.5 mm. In certain embodiments, the size is from about 0.5 mm to about 10 mm, about 1 mm to about 8 mm or about 1.5 mm to about 5 mm.

In certain embodiments, the active agent is suspended in the polymer composition.

In certain embodiments, the active agent is homogenousely dispersed in the polymer composition.

In certain embodiments, the extrusion process is performed without solvent (e.g., water), In certain embodiments a solvent is used in an amount of less than about 10% w/w, less than about 5% w/w or less than about 1% w/w. The solvent may be, e.g., water or an oil. An oil may result in an increased release rate for lipophilic active agents. The oil may a biocompatible vegetable oil, a synthetic oil or a mineral oil, a liquid fatty acid or triglyceride composition, or it may be a hydrophobic biodegradable liquid polymer, or combinations thereof. In certain embodiments, the oil may comprise triethyl citrate, acetyl triethyl citrate (ATEC), acetyl tributyl citrate (ATBC), α-tocopherol (vitamin E), α-tocopherol acetate; plant or vegetable oils such as sesame oil, olive oil, soybean oil, sunflower oil, coconut oil, canola oil, rapeseed oil, nut oils such as hazelnut, walnut, pecan, almond, cottonseed oil, corn oil, safflower oil, linseed oil, etc., ethyl oleate, castor oil and derivatives thereof (Cremophor®), lipids being liquid at 37° C. or lower, such as saturated or unsaturated fatty acids, monoglycerides, diglycerides, triglycerides (Myglyolsg), phospholipids, glycerophospholipids, sphingolipids, sterols, prenols, polyketides, hydrophobic biodegradable liquid polymers (such as low molecular weight PLGA, PGA or PLA etc.), low melting point waxes such as plant, animal or synthetic waxes, lanolin, jojoba oil, or combinations thereof.

In certain embodiments, the content uniformity of the unit dose insert or implant is within 10%, with 5% or within 1%.

In certain embodiments, the persistence of the dosage form is from about 1 day to about 1 year, about 2 days to about 9 months or about 7 days to about 6 months after administration. This can be increased or decreased based on factors such as crosslinking.

In certain embodiments, the polymorphic form of the active agent does not change or does not substantially change. In certain embodiments, the purity of the active agent after curing is greater than 99%, greater than 99.5% or greater than 99.9% as compared to the active agent prior to extrusion. Purity is measured by chemical degradation of the active agent.

In certain embodiments, the active agent has a median (D50) particle diameter of less than about 100 μm, less than about 50 μm, less than about 25 μm, or less than about 10 μm.

In certain embodiments, the active agent has a D50 particle size of less than about 10 μm and/or a D99 particle size of less than about 50 or a D90 particle size of about 5 μm or less and/or a D98 particle size of about 10 μm or less.

In certain embodiments, the polymer composition comprises polyethylene glycol,

polyethylene oxide, polypropylene oxide, polyvinyl alcohol, poly (vinylpyrrolidinone), polylactic acid, polylactic-co-glycolic acid, random or block copolymers, polycaprolactone, ethylenevinyl acetate or combinations or mixtures of any of these, or one or more units of polyaminoacids, glycosaminoglycans, polysaccharides, proteins, cellulosic polymers (e.g., hydroxypropylmethylcellulose), povidone, poloxamer, acrlic polymners (e.g., polymethacrylates) or a combination thereof.

In certain embodiments, the polymer composition comprises an electrophilic group-containing multi-arm polyethylene glycol.

In certain embodiments, the polymer composition further comprises a nucleophilic group-containing crosslinking agent.

In certain embodiments, the crosslinking agent contains amine groups.

In certain embodiments, the electrophilic group-containing multi-arm-polymer precursor is 4a20kPEG-SG and the crosslinking agent is trilysine acetate.

In certain embodiments, the polymer composition further comprises a visualization agent. Ion other embodiments, the polymer composition further comprises a radiopaque agent, e.g., for x-ray or magnetic resonance imaging.

In certain embodiments, the visualization agent is a fluorophore.

In certain embodiments, the ocular insert or implant is suitable for intracanalicular, suprachoroidal, intracameral, fornix or intravitreal administration. The administration can be manually, with an insert or implant tool or device or by injection.

In certain embodiments, the active agent is selected from dexamethasone, travoprost, cyclosporine, axitinib, bupivacaine, ropivacaine, lidocaine as well as various polymorphic, co-crystal, salt or prodrug forms.

In certain embodiments, the extrusion process excludes water.

In certain embodiments, the present invention is directed to an ocular insert or implant prepared by a method as disclosed herein.

In certain embodiments, the present invention is directed to a method of treating an ocular disease comprising administering an ocular insert or implant as disclosed herein.

One or more of these objects of the present invention and others are solved by one or more embodiments of the invention as disclosed and claimed herein.

### The Implant and the Active Principle:

In certain embodiments, the inserts or implants disclosed herein are suitable for ocular delivery to a route selected from, e.g., punctal, intravitreal, subconjunctival, intrascleral, subretinal, suprachoroidal, periocular, peribulbar, retrobulbar, intracorneal, posterior sub-tenon's delivery, anterior sub-tenon's delivery, cul-de-sac delivery, or fornix delivery. The administration can be, e.g., by injection with a needle or insertion with a delivery device into the selected ocular delivery route.

The needle can be a gauge selected from, e.g., 18 gauge, 19 gauge, 20 gauge, 21 gauge, 22 gauge, 23 gauge, 24 gauge, 25 gauge, 26 gauge, 27 gauge, 28 gauge, 29 gauge, 30 gauge, 31 gauge, 32 gauge or 33 gauge.

In certain embodiments, the administration can be with a modified device as described in U.S. Pat. Nos. 8,808,225; 10,722,396; 10,390,901; 10,188,550; 9,956,114; 9,931,330; U.S. Patent Application Publication No. 2019/0290485; U.S. Patent Application Publication No. 2019/0000669; and U.S. Patent Application Publication No. 2018/0042767.

In alternative embodiments where the ocular delivery route is accessible from the exterior of the eye, the administration can optionally be performed without a needle, e.g., manually or with the aid of tweezer, applicator or other delivery aid.

The active agent administered by the implants of the present invention can, e.g., have an aqueous solubility of less than about 2,000 μg/mL, less than about 1,500 μg/mL, less than about 1,000 μg/mL, less than about 800 μg/mL, less than about 600 μg/mL, less than about 500 μg/mL, less than about 400 μg/mL, less than about 300 μg/mL, less than about 200 μg/mL, less than about 100 μg/mL, less than about 75 μg/mL, less than about 50 μg/mL, less than about 25 μg/mL, less than about 10 μg/mL, less than about 5 μg/mL, less than about 1 μg/mL, less than about 0.5 μg/mL, less than about 0.4 μg/mL, less than about 0.3 μg/mL, less than about 0.2 μg/mL or less than about 0.1 μg/mL.

In other embodiments, the active agents administered by the devices of the present invention can have an aqueous solubility of sparingly soluble (30-100 parts solvent needed for 1 part solute), slightly soluble (100-1,000 parts solvent needed for 1 part solute), very slightly soluble (1,000-10,000 parts solvent needed for 1 part solute) or practically insoluble or insoluble (>10,000 parts solvent needed for 1 part solute) as described in Remington, The Science and Practice of Pharmacy 22nd Edition 2012.

Ocular diseases that can be treated with the implants and methods of the present invention may include any ophthalmic condition such as front of the eye conditions or back of the eye conditions.

Front of the eye conditions may be associated with cellular or subcellular components of the front of the eye anatomy such as the acellular tear film layer and its corresponding lipid aqueous mucin components. Front of the eye conditions may also be associated with the upper and lower eyelids including conditions of the meibomian gland and its corresponding cellular and tissue components such as the muscle, lipid producing holocrine, exocrine and endocrine glands and vascular and connective tissue components; and the conjunctiva and its corresponding cells including goblet cells, fibroblast cells, vascular and component blood cells. Front of the eye conditions may further be associated with the corneal layers of the eye including the layers of epithelial cells, stromal cells and fibroblasts, corneal endothelial cells, corneal nerve its associated cells and ground substances. Front of the eye conditions may also include inflammation, diffuse lamellar keratitis, corneal diseases, edemas, or opacifications with an exudative or inflammatory component, eye conditions related to systemic autoimmune diseases, ocular surface disorders from dry eye (e.g., keratoconjunctivitis such as vernal keratoconjunctivitis, atopic keratoconjunctivitis and sicca keratoconjunctivitis), lid margin diseases, meibomian gland conditions, dysfunctional tear syndromes, anterior and posterior blepharitis, staphylococcal blepharitis, microbial infection, conjunctivitis (e.g., persistent allergic, giant papillary, seasonal intermittent allergic, perennial allergic, toxic and infectious conjunctivitis), conjunctival edema, anterior uveitis, inflammatory conditions, edema, genetic conditions of the cornea (e.g., corneal dystrophies such as keratoconus, posterior polymorphous dystrophy), Fuchs' dystrophies, aphakic and pseudophakic bullous keratopathy, scleral diseases, ocular cicatricial pemphigoid and pterygium.

Back of the eye conditions may be related to cellular or subcellular components of the back of the eye anatomy including the retina and all of the cells of the layers of the retina such as outer and inner photoreceptor layers, nuclear cell layers, amacrine and ganglion cells, macula, fovea, and vitreous. Additional components of the back of the eye include the ciliary body, iris, uvea and the retinal pigment cells. Back of the eye conditions may include conditions of the optic nerve (including corresponding cellular and sub cellular components such as the axons and associated innervations), glaucomas (e.g., primary open angle glaucoma, acute and chronic closed angle glaucoma and secondary glaucomas), myopic retinopathies, macular edema (including clinical macular edema or angiographic cystoid macular edema arising from conditions such as diabetes, exudative macular degeneration and macular edema associated with laser treatment of the retina), diabetic retinopathy, age-related macular degeneration, retinopathy of prematurity, retinal ischemia and choroidal neovascularization, genetic disease of the retina, pars planitis, Posner Schlossman syndrome, Bechet's disease, Vogt-Koyanagi-Harada syndrome, hypersensitivity reactions, toxoplasmosis chorioretinitis, inflammatory pseudotumor of the orbit, chemosis, conjunctival venous congestion, periorbital cellulitis, acute dacryocystitis, non-specific vasculitis, sarcoidosis, and cytomegalovirus infection.

Specific active agents that can be utilized in the implants and methods of the present invention include but are not limited to immunosuppressants, complement protein C5 agents (e.g., eculizumab or avacincaptad pegol), steroids, anti-inflammatories such as steroidal and non-steroidal anti-inflammatories (e.g., COX1 or COX 2 inhibitors), antivirals, antibiotics, anti-glaucoma agents, anti-VEGF agents, analgesics and combinations thereof.

Immunosuppressants include but are not limited to cyclosporine, mTOR inhibitors (e.g., rapamycin, tacrilimus, temsirolimus, sirolimus, everolimus, KU-0063794, WYE-354, AZD8055, metformin, or Torin-2), cyclophosphamide, atoposide, thiotepa, methotrexate, azathioprine, mercaptopurine, interferons, infliximab, etanercept, mycophenolate mofetil, 15-deoxyspergualin, thalidomide, glatiramer, leflunomide, vincristine, cytarabine, pharmaceutically acceptable salts thereof and combinations thereof.

Non-steroidal anti-inflammatory compounds include inhibitors of the cyclooxygenase (COX) enzyme such as cyclooxygenase-1 (COX-1) and cyclooxygenase-2 (COX-2) isozymes. General classes of non-steroidal anti-inflammatory compounds include salicylates, propionic acid derivatives, acetic acid derivatives, enolic acid derivatives, and anthranilic acid derivatives. Examples of non-steroidal anti-inflammatory compounds include acetylsalicylic acid, diflunisal, salsalate, ibuprofen, dex-ibuprofen, naproxen, nepafenac, fenoprofen, ketoprofen, dex-ketoprofen, flurbiprofen, oxaprozin, loxoprofen, indomethacin, tolmetin, sulindac, etodolac, ketorolac, diclofenac, aceclofenac, nabumetone, piroxicam, tenoxicam, tenoxicam, loroxicam, phenylbutazone, mefenamic acid, meclofenamic acid, flufenamic acid, tolfenamic acid, celecoxib, pharmaceutically acceptable salts thereof and combinations thereof.

Anti-inflammatory agents that may be utilized in the implants and methods of the present invention may include agents that target inflammatory cytokines such as TNFα, IL-1, IL-4, IL-5 or IL-17, or CD20. Such agents may include etanercept, infliximab, adalimumab, daclizumab, rituximab, tocilizumab, certolizumab pegol, golimumab, pharmaceutically acceptable salts thereof and combinations thereof.

Analgesics that may be utilized in the implants and methods of the present invention include acetaminophen, acetaminosalol, aminochlorthenoxazin, acetylsalicylic 2-amino-4-picoline acid, acetyl salicyl salicylic acid, anileridine, benoxaprofen, benzylmorphine, 5-bromosalicylic acetate acid, bucetin, buprenorphine, butorphanol, capsaicin, cinchophen, ciramadol, clometacin, clonixin, codeine, desomorphine, dezocine, dihydrocodeine, dihydromorphine, dimepheptanol, dipyrocetyl, eptazocine, ethoxazene, ethylmorphine, eugenol, floctafenine, fosfosal, glafenine, hydrocodone, hydromorphone, hydroxypethidine, ibufenac, p-lactophenetide, levorphanol, meptazinol, metazocine, metopon, morphine, nalbuphine, nicomorphine, norlevorphanol, normorphine, oxycodone, oxymorphone, pentazocine, phenazocine, phenocoll, phenoperidine, phenylbutazone, phenylsalicylate, phenylramidol, salicin, salicylamide, tiorphan, tramadol, diacerein, actarit, pharmaceutically acceptable salts thereof and combinations thereof.

Antibiotic that may be utilized in the implants and methods of the present invention include aminoglycosides, penicillins, cephalosporins, fluoroquinolones, macrolides, and combinations thereof. Aminoglycosides may include tobramycin, kanamycin A, amikacin, dibekacin, gentamicin, sisomicin, netilmicin, neomycin B, neomycin C, neomycin E, streptomycin, paramomycin, pharmaceutically acceptable salts thereof and combinations thereof. Penicillins may include amoxicillin, ampicillin, bacampicillin, carbenicillin, cloxacillin, dicloxacillin, flucloxacillin, mezlocillin, nafcillin, oxacillin, penicillin G, penicillin V, piperacillin, pivampicillin, pivmecillinam, ticarcillin, pharmaceutically acceptable salts thereof and combinations thereof. Cephalosporins may include cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime, cefluprenam, cefoselis, cefozopran, cefpirome, cefquinome, ceftobiprole, ceftaroline, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, cefuracetime, ceftioxide, pharmaceutically acceptable salts thereof and combinations thereof. Fluoroquinolones may include ciprofloxacin, levofloxacin, gatifloxacin, moxifloxacin, ofloxacin, norfloxacin, pharmaceutically acceptable salts thereof and combinations thereof. Macrolides may include azithromycin, erythromycin, clarithromycin, dirithromycin, oxithromycin, telithromycin, pharmaceutically acceptable salts thereof and combinations thereof.

Antivirals that may be utilized in the implants and methods of the present invention include nucleoside reverse transcriptase inhibitors, non-nucleoside reverse transcriptase inhibitors, fusion inhibitors, integrase inhibitors, nucleoside analogs, protease inhibitors, and reverse transcriptase inhibitors. Examples of antiviral agents include, but are not limited to, abacavir, aciclovir, acyclovir, adefovir, amantadine, amprenavir, ampligen, arbidol, atazanavir, boceprevir, cidofovir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, interferon, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramiding saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine, pharmaceutically acceptable salts thereof and combinations thereof.

Steroidal anti-inflammatory agents that may be utilized in the implants and methods of the present invention include dexamethasone, budensonide, triamcinolone, hydrocortisone, loteprednol, prednisolone, mometasone, fluticasone, rimexolone, fluorometholone, beclomethasone, flunisolide, pharmaceutically acceptable salts thereof and combinations thereof.

Anti-glaucoma agents that may be utilized in the implants and methods of the present invention include beta-blockers such as atenolol propranolol, metipranolol, betaxolol, carteolol, levobetaxolol, levobunolol timolol, pharmaceutically acceptable salts thereof and combinations thereof adrenergic agonists or sympathomimetic agents such as epinephrine, dipivefrin, clonidine, aparclonidine, brimonidine, pharmaceutically acceptable salts thereof and combinations thereof parasympathomimetics or cholingeric agonists such as pilocarpine, carbachol, phospholine iodine, physostigmine, pharmaceutically acceptable salts thereof and combinations thereof carbonic anhydrase inhibitor agents, including topical or systemic agents such as acetozolamide, brinzolamide, dorzolamide; methazolamide, ethoxzolamide, dichlorphenamide, pharmaceutically acceptable salts thereof and combinations thereof; mydriatic-cycloplegic agents such as atropine, cyclopentolate, succinylcholine, homatropine, phenylephrine, scopolamine, tropicamide, pharmaceutically acceptable salts thereof and combinations thereof prostaglandins such as prostaglandin F2 alpha, antiprostaglandins, prostaglandin precursors, or prostaglandin analog agents such as bimatoprost, latanoprost, travoprost, unoprostone, tafluprost, pharmaceutically acceptable salts thereof and combinations thereof.

Anti-VEGF agents that may be utilized in the implants and methods of the present invention include bevacizumab, pegaptanib, ranibizumab, brolucizumab, pharmaceutically acceptable salts thereof and combinations thereof.

In certain embodiments, the active principle contained in an implant of this aspect of the invention is a TKI. Examples for suitable TKIs are axitinib, sorafenib, sunitinib, nintedanib, pazopanib, regorafenib, cabozantinib, and vandetanib. In particular embodiments, the TKI used in this and other aspects of the present invention is axitinib. Details on axitinib, its chemical structure, polymorphs, solvates, salts etc. and its properties such as solubility are provided above in the definitions section.

In certain embodiments, the active agent is dexamethasone and the insert or implant provided an in-vitro release of dexamethasone of one or more of (i) at 1 hour of from about 30% to about 70% or about 40% to about 65%; (ii) at 2 hours of from about 60% to about 90% or about 65% to about 85%.; or (iii) at 4 hours of greater than about 85% or greater than about 90%. The in-vitro release is measured at 37° C. in water with Ultra Performance Liquid Chromatography using an Acquity BEH C8 Column or equivalent; or by pH4 phosphate buffered saline (PBS) on a Mettler Toledo UV5 Spectrometer or equivalent.

### The Polymer Composition or Network:

The hydrogel may be formed from precursors having functional groups that form crosslinks to create a polymer network. These crosslinks between polymer strands or arms may be chemical (i.e., may be covalent bonds) and/or physical (such as ionic bonds, hydrophobic association, hydrogen bridges etc.) in nature.

The polymer network may be prepared from precursors, either from one type of precursor or from two or more types of precursors that are allowed to react. Precursors are chosen in consideration of the properties that are desired for the resultant hydrogel. There are various suitable precursors for use in making the hydrogels. Generally, any pharmaceutically acceptable and crosslinkable polymers forming a hydrogel may be used for the purposes of the present invention. The hydrogel and thus the components incorporated into it, including the polymers used for making the polymer network, should be physiologically safe such that they do not elicit e.g. an immune response or other adverse effects. Hydrogels may be formed from natural, synthetic, or biosynthetic polymers.

Natural polymers may include glycosaminoglycans, polysaccharides (e.g. dextran), polyaminoacids and proteins or mixtures or combinations thereof, while this list is not intended to be limiting.

Synthetic polymers may generally be any polymers that are synthetically produced from a variety of feedstocks by different types of polymerization, including free radical polymerization, anionic or cationic polymerization, chain-growth or addition polymerization, condensation polymerization, ring-opening polymerization etc. The polymerization may be initiated by certain initiators, by light and/or heat, and may be mediated by catalysts. Synthetic polymers may in certain embodiments be used to lower the potential of allergies in dosage forms that do not contain any ingredients from human or animal origin.

Generally, for the purposes of the present invention one or more synthetic polymers of the group comprising one or more units of polyethylene glycol (PEG), polyethylene oxide, polypropylene oxide, polyvinyl alcohol, poly (vinylpyrrolidinone), polylactic acid, polylactic-co-glycolic acid, random or block copolymers or combinations/mixtures of any of these can be used, while this list is not intended to be limiting.

To form covalently crosslinked polymer networks, the precursors may be covalently crosslinked with each other. In certain embodiments, precursors with at least two reactive centers (for example, in free radical polymerization) can serve as crosslinkers since each reactive group can participate in the formation of a different growing polymer chain.

The precursors may have biologically inert and hydrophilic portions, e.g., a core. In the case of a branched polymer, a core refers to a contiguous portion of a molecule joined to arms that extend from the core, where the arms carry a functional group, which is often at the terminus of the arm or branch. Multi-armed PEG precursors are examples of such precursors and are used in particular embodiments of the present invention as further disclosed herein.

A hydrogel for use in the present invention can be made e.g. from one multi-armed precursor with a first (set of) functional group(s) and another (e.g. multi-armed) precursor having a second (set of) functional group(s). By way of example, a multi-armed precursor may have hydrophilic arms, e.g., polyethylene glycol units, terminated with primary amines (nucleophile), or may have activated ester end groups (electrophile). The polymer network according to the present invention may contain identical or different polymer units crosslinked with each other.

The precursors may be high-molecular weight components (such as polymers having functional groups as further disclosed herein) or low-molecular weight components (such as low-molecular amines, thiols, esters etc. as also further disclosed herein).

Certain functional groups can be made more reactive by using an activating group. Such activating groups include (but are not limited to) carbonyldiimidazole, sulfonyl chloride, aryl halides, sulfosuccinimidyl esters, N-hydroxysuccinimidyl (abbreviated as “NETS”) ester, succinimidyl ester, benzotriazolyl ester, thioester, epoxide, aldehyde, maleimides, imidoesters, acrylates and the like. The NETS esters are useful groups for crosslinking with nucleophilic polymers, e.g., primary amine-terminated or thiol-terminated polyethylene glycols. An NETS-amine crosslinking reaction may be carried out in aqueous solution and in the presence of buffers, e.g., phosphate buffer (pH 5.0-7.5), triethanolamine buffer (pH 7.5-9.0), borate buffer (pH 9.0-12), or sodium bicarbonate buffer (pH 9.0-10.0).

In certain embodiments, each precursor may comprise only nucleophilic or only electrophilic functional groups, so long as both nucleophilic and electrophilic precursors are used in the crosslinking reaction. Thus, for example, if a crosslinker has only nucleophilic functional groups such as amines, the precursor polymer may have electrophilic functional groups such as N-hydroxysuccinimides. On the other hand, if a crosslinker has electrophilic functional groups such as sulfosuccinimides, then the functional polymer may have nucleophilic functional groups such as amines or thiols. Thus, functional polymers such as proteins, poly (allyl amine), or amine-terminated di-or multifunctional poly(ethylene glycol) can be also used to prepare the polymer network of the present invention.

In one embodiment of the present invention a precursor for the polymer network forming the hydrogel in which the glucocorticoid is dispersed to form the insert or implant according to the present invention has about 2 to about 16 nucleophilic functional groups each (termed functionality), and in another embodiment a precursor has about 2 to about 16 electrophilic functional groups each (termed functionality). Reactive precursors having a number of reactive (nucleophilic or electrophilic) groups as a multiple of 4, thus for example 4, 8 and 16 reactive groups, are particularly suitable for the present invention. However, any number of functional groups, such as including any of 2, 3, 4, 5, 6, 7, 8, 9, 10, 11, 12, 13, 14, 15, or 16 groups, is possible for precursors to be used in accordance with the present invention, while ensuring that the functionality is sufficient to form an adequately crosslinked network.

### Peg Hydrogels:

In certain embodiments of the present invention, the polymer network forming the hydrogel contains polyethylene glycol (“PEG”) units. PEGs are known in the art to form hydrogels when crosslinked, and these PEG hydrogels are suitable for pharmaceutical applications e.g. as matrix for drugs intended to be administered to any part of the human or animal body.

The polymer network of the hydrogel inserts or implants of the present invention may comprise one or more multi-arm PEG units having from 2 to 10 arms, or from 4 to 8 arms, or 4, 6, 7 or 8 arms. In certain embodiments, the PEG units used in the hydrogel of the present invention have 4 arm. In certain embodiments, the PEG units used in the hydrogel of the present invention have 8 arms. In certain embodiments, PEG units having 4 arms and PEG units having 8 arms are used in the hydrogel of the present invention. In certain particular embodiments, one or more 4-armed PEGs is/are utilized.

The number of arms and the molecular weight of the PEG used contributes to controlling the flexibility or softness of the resulting hydrogel. For example, hydrogels formed by crosslinking 4-arm PEGs are generally softer and more flexible than those formed from 8-arm PEGs of the same molecular weight. Also, higher molecular weights, for a given number of arms, typically increase softness. In particular, if stretching the hydrogel prior to (or also after) drying as disclosed herein below in the section relating to the manufacture of the insert or implant is desired, a more flexible hydrogel may be used, such as a 4-arm PEG, optionally in combination with another multi-arm PEG, such as an 8-arm PEG as disclosed above, or another (different) 4-arm PEG.

In certain embodiments of the present invention, polyethylene glycol units used as precursors have a number average molecular weight (Mn) in the range from about 2,000 to about 100,000 Daltons, or in a range from about 10,000 to about 60,000 Daltons, or in a range from about 15,000 to about 50,000 Daltons. These polymers typically have narrow polydispersity, e.g., Mw/Mn of <1.1. In certain particular embodiments the polyethylene glycol units have an average molecular weight in a range from about 10,000 to about 40,000 Daltons. In specific embodiments, the polyethylene glycol units used for making the hydrogels according to the present invention have an average molecular weight of about 20,000 Daltons. Polyethylene glycol precursors of different molecular weight may be combined with each other. When referring herein to a PEG material having a particular average molecular weight, such as about 20,000 Daltons, a variance of ±10% is intended to be included, i.e., referring to a material having an average molecular weight of about 20,000 Daltons also refers to such a material having an average molecular weight of about 18,000 to about 22,000 Daltons. As used herein, the abbreviation “k” in the context of the molecular weight refers to 1,000 Daltons, i.e., “20 k” means 20,000 Daltons.

In a 4-arm (“4a”) PEG, in certain embodiments each of the arms may have an average arm length (or molecular weight) of the total molecular weight of the PEG divided by 4. A 4a20kPEG precursor, which is a particularly suitably precursor for use in the present invention thus has 4 arms with an average molecular weight of about 5,000 Daltons each and a total molecular weight of 20,000 Daltons. An 8a20 k PEG precursor, which could also be used in combination with or alternatively to the 4a20kPEG precursor in the present invention, thus has 8 arms (“8a”) each having an average molecular weight of 2,500 Daltons and a total molecular weight of 20,000 Daltons. Longer arms may provide increased flexibility as compared to shorter arms. PEGs with longer arms may swell more as compared to PEGs with shorter arms. A PEG with a lower number of arms also may swell more and may be more flexible than a PEG with a higher number of arms. In certain particular embodiments, only a 4-arm PEG precursor is utilized in the present invention. In certain other embodiments, a combination of a 4-arm PEG precursor and an 8-arm precursor is utilized in the present invention. In addition, longer PEG arms have higher melting temperatures when dry, which may provide more dimensional stability during storage.

In certain embodiments, electrophilic end groups for use with PEG precursors for preparing the hydrogels of the present invention are N-hydroxysuccinimidyl (NHS) esters, including but not limited to NHS dicarboxylic acid esters such as the succinimidylmalonate group, succinimidylmaleate group, succinimidylfumarate group, “SAZ” referring to a succinimidylazelate end group, “SAP” referring to a succinimidyladipate end group, “SG” referring to a succinimidylglutarate end group, “SS” referring to a succinimidylsuccinate end group and “SGA referring to a succinimidylglutaramide end group.

In certain embodiments, nucleophilic end groups for use with electrophilic group-containing PEG precursors for preparing the hydrogels of the present invention are amine (denoted as “NH₂”) end groups. Thiol (—SH) end groups or other nucleophilic end groups are also possible.

In certain preferred embodiments of the present invention, 4-arm PEGs with an average molecular weight of about 20,000 Daltons and electrophilic end groups as disclosed above (such as the SAZ, SAP, SG and SS end groups, particularly the SG end group) are crosslinked for forming the polymer network and thus the hydrogel according to the present invention. Suitable PEG precursors are available from a number of suppliers, such as Jenkem Technology and others.

Reactions of e.g. nucleophilic group-containing crosslinkers and electrophilic group-containing PEG units, such as reaction of amine group-containing crosslinkers with activated ester-group containing PEG units, result in a plurality of PEG units being crosslinked by a hydrolyzable linker having the formula:

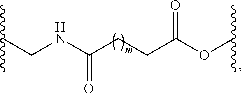

wherein m is an integer from 0 to 10, and specifically is 1, 2, 3, 4, 5, 6, 7, 8, 9, or 10. For a SAZ-end group, m would be 6, for a SAP-end group, m would be 3, for a SG-end group, m would be 2 and for an SS-end group, m would be 1.

In certain embodiments, the polymer precursors used for forming the hydrogel according to the present invention may be selected from 4a20kPEG-SAZ, 4a20kPEG-SAP, 4a20kPEG-SG, 4a20kPEG-SS, 8a20kPEG-SAZ, 8a20kPEG-SAP, 8a20kPEG-SG, 8a20kPEG-SS, or mixtures thereof, with one or more PEG- or lysine based-amine groups selected from 4a20kPEG-NH₂, 8a20kPEG-NH 2, and trilysine, or a trilysine salt or derivative, such as trilysine acetate. In certain embodiments, the salt form is protective and is removed in water and the cross-linking reaction will occur when the polymer is melted and mixed. If a non-salt is used the cross-linking can occur more rapidly. In certain embodiments, an additional entry/feed port of the extruder can introduce a crosslinker just before extrusion

In certain embodiments, the SG end group is utilized in the present invention. This end group may provide for a shorter time until the hydrogel is biodegraded in an aqueous environment such as in the tear fluid, when compared to the use of other end groups, such as the SAZ end group, which provides for a higher number of carbon atoms in the linker and may thus be more hydrophobic and therefore less prone to ester hydrolysis than the SG end group.

In particular embodiments, a 4-arm 20,000 Dalton PEG precursor having a SG end group (as defined above), is crosslinked with a crosslinking agent having one or more reactive amine end groups. This PEG precursor is abbreviated herein as 4a20kPEG-SG. A schematic chemical structure of 4a20kPEG-SG is reproduced below:

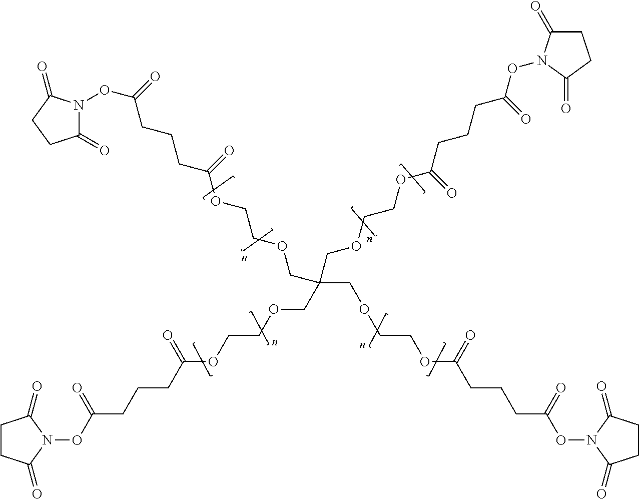

In this formula, n is determined by the molecular weight of the respective PEG-arm.

In certain particular embodiments, the crosslinking agent (herein also referred to as “crosslinker”) used is a low-molecular weight component containing nucleophilic end groups, such as amine or thiol end groups. In certain embodiments, the nucleophilic group-containing crosslinking agent is a small molecule amine with a molecular weight below 1,000 Da. In certain embodiments, the nucleophilic-group containing crosslinking agent comprises two, three or more primary aliphatic amine groups. Suitable crosslinking agents for use in the present invention are (without being limited to) spermine, spermidine, lysine, dilysine, trilysine, tetralysine, polylysine, ethylenediamine, polyethylenimine, 1,3-diaminopropane, 1,3-diaminopropane, diethylenetriamine, trimethylhexamethylenediamine, 1,1,1-tris(aminoethyl)ethane, their pharmaceutically acceptable salts, hydrates or other solvates and their derivatives such as conjugates (as long as sufficient nucleophilic groups for crosslinking remain present), and any mixtures thereof. A particular crosslinking agent for use in the present invention is trilysine or a trilysine salt or derivative, such as trilysine acetate. Other low-molecular weight multi-arm amines may be used as well. The chemical structure of trilysine is reproduced below:

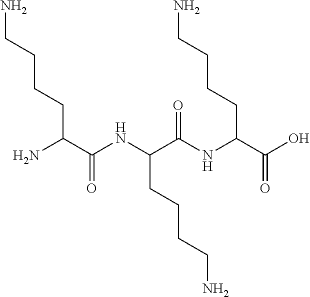 [SMILES: NCCCCC(N)C(=O)NC(CCCCN)C(=O)NC(CCCCN)C(=O)O]

In very particular embodiments of the present invention, a 4a20kPEG-SG precursor is reacted with trilysine acetate, to form the polymer network.

In certain embodiments, the nucleophilic group-containing crosslinking agent is bound to or conjugated with a visualization agent. Fluorophores such as fluorescein, rhodamine, coumarin, and cyanine can be used as visualization agents as disclosed herein. In specific embodiments of the present invention, fluorescein is used as the visualization agent. The visualization agent may be conjugated with the crosslinking agent e.g. through some of the nucleophilic groups of the crosslinking agent. Since a sufficient amount of the nucleophilic groups are necessary for crosslinking, “conjugated” or “conjugation” in general includes partial conjugation, meaning that only a part of the nucleophilic groups are used for conjugation with the visualization agent, such as about 1% to about 20%, or about 5% to about 10%, or about 8% of the nucleophilic groups of the crosslinking agent may be conjugated with a visualization agent. In specific embodiments, the crosslinking agent is trilysine acetate and is conjugated with fluorescein.

In other embodiments, the visualization agent may also be conjugated with the polymer precursor, e.g. through certain reactive (such as electrophilic) groups of the polymer precursors. In certain embodiments, the crosslinking agent itself or the polymer precursor itself may contain an e.g. fluorophoric or other visualization-enabling group.

In the present invention, conjugation of the visualization agent to either the polymer precursor(s) or to the crosslinking agent as disclosed below is intended to keep the visualization agent in the hydrogel while the active agent is released into the tear fluid, thus allowing confirmation of insert presence within the canaliculus by a convenient, non-invasive method.

In certain embodiments, the molar ratio of the nucleophilic and the electrophilic end groups reacting with each other is about 1:1, i.e., one amine group is provided per one electrophilic, such as SG, group. In the case of 4a20kPEG-SG and trilysine (acetate) this results in a molar ratio of the two components of about 1:1 as the trilysine has four primary amine groups that may react with the electrophilic SG ester group. However, an excess of either the electrophilic (e.g. the NHS end groups, such as the SG) end group precursor or of the nucleophilic (e.g. the amine) end group precursor may be used. In particular, an excess of the nucleophilic, such as the amine end group containing precursor or crosslinking agent may be used. In certain embodiments, the molar ratio of the electrophilic group containing precursor to the nucleophilic group-containing crosslinking agent, such as the molar ratio of 4a20kPEG-SG to trilysine acetate, is from about 1:2 to about 0.5:1, or from about 1:2 to about 2:1.

Finally, in alternative embodiments the amine linking agent can also be another PEG precursor with the same or a different number of arms and the same or a different arm length (average molecular weight) as the 4a20kPEG-SG, but having terminal amine groups, i.e., 4a20kPEG-NEH₂.

### Additional Ingredients:

The insert or implant of the present invention may contain, in addition to the polymer units forming the polymer network as disclosed above and the active principle, other additional ingredients. Such additional ingredients are for example salts originating from buffers used during the preparation of the hydrogel, such as phosphates, borates, bicarbonates, or other buffer agents such as triethanolamine. In certain embodiments of the present invention sodium phosphate buffers (specifically, mono- and dibasic sodium phosphate) are used.

In a specific embodiment, the insert or implant of the present invention is free of anti-microbial preservatives or at least does not contain a substantial amount of anti-microbial preservatives.

In a further specific embodiment, the insert or implant of the present invention does not contain any ingredients of animals or human origin but only contains synthetic ingredients.

In certain embodiments, the inserts or implants of the present invention contain a visualization agent. Visualization agents to be used according to the present invention are all agents that can be conjugated with the components of the hydrogel or can be entrapped within the hydrogel, and that are visible, or may be made visible when exposed e.g. to light of a certain wavelength, or that are constrast agents. Suitable visualization agents for use in the present invention are (but are not limited to) e.g. fluoresceins, rhodamines, coumarins, cyanines, europium chelate complexes, boron dipyromethenes, benzofrazans, dansyls, bimanes, acridines, triazapentalenes, pyrenes and derivatives thereof. Such visualization agents are commercially available e.g. from TCI. In certain embodiments the visualization agent is a fluorophore, such as fluorescein. Visualization of the fluorescein-containing insert or implant is possible by illumination with blue light. The fluorescein in the intracanalicular insert illuminates when excited with blue light enabling confirmation of insert presence. In specific embodiments, the visualization agent is conjugated with one of the components forming the hydrogel. For example, the visualization agent, such as fluorescein, is conjugated with the crosslinking agent, such as the trilysine or trilysine salt or derivate (e.g. the trilysine acetate), or with the PEG-component e.g. by means of reacting NHS-fluorescein with trilysine acetate. Conjugation of the visualization agent prevents the visualization agent from being eluted or released out of the insert.

In other embodiments, the formulations may include a plasticizer (e.g., stearic acid or glyceryl behenate) or pore former (e.g., mannitol, sorbitol or calcium carbonate). The inclusion of a plasticizer may help the formulation to be more extrudable/flowable and pore formers may be used to to allow for greater surface area upon dissolution and faster release rate.

### Formulation:

In certain embodiments, inserts or implants according to the present invention comprise an active agent, a polymer network made from one or more polymer precursors as disclosed herein in the form of a hydrogel, and optional additional components such as visualization agents, salts etc. remaining in the insert or implant from the production process (such as phosphate salts used as buffers etc.). In particular embodiments, the insert or implant is preservative-free.

In some embodiments, the inserts or implants according to the present invention in a dry state contain from about 5% to about 80% by weight active agent, and from about 15% to about 95% by weight polymer units, such as those disclosed above. In other embodiments, the inserts or implants according to the present invention in a dry state contain from about 30% to about 70% by weight active agent, and from about 25% to about 60% by weight polymer units, such as those disclosed above. In further embodiments, the inserts or implants according to the present invention in a dry state contain from about 30% to about 60% by weight active agent, and from about 30% to about 60% by weight polymer units, such as those disclosed above.

In certain embodiments, the inserts or implants according to the present invention may contain in a dry state about 0.1% to about 1% by weight visualization agent, such as fluorescein. Also in certain embodiments, the inserts or implants according to the present invention may contain in a dry state about 0.5% to about 5% by weight of one or more buffer salt(s) (separately or taken together). In such embodiments, the insert or implant in a dry state may contain from about 0.01% to about 2% by weight or from about 0.05% to about 0.5% by weight of a surfactant.

The amounts of the active agent and the polymer(s) may be varied, and other amounts of the active agent and the polymer hydrogel than those disclosed herein may also be used to prepare inserts or implants according to the invention.

In certain embodiments, the maximum amount (in weight %) of drug within the formulation is about two times the amount of the polymer (e.g., PEG) units, but may be higher in certain cases, as long as the mixture comprising e.g., the precursors, visualization agent, buffers and drug (in the state before the hydrogel has gelled completely) can be uniformly extruded and/or the hydrogel is still sufficiently stretchable as disclosed herein, and/or sufficiently increases in diameter upon hydration as also disclosed herein.

In certain embodiments, solid contents of about 20% to about 50% (w/v) (wherein “solids” means the combined weight of polymer precursor(s), optional visualization agent, salts and the drug in solution) are utilized for forming the hydrogel of the inserts or implants according to the present invention.

In certain embodiments, the water content of the hydrogel in a dry (dehydrated/dried) state may be low, such as not more than about 1% by weight of water. The water content may in certain embodiments also be lower than that, possibly no more than about 0.25% by weight or even no more than about 0.1% by weight.

### Dimensions of the Insert or Implant and Dimensional Change Upon Hydration Through Stretching:

The dried insert or implant may have different geometries, depending on the method of manufacture, such as the dimensions and shape of a mold or extrusion die into or from which the mixture comprising the hydrogel precursors including the glucocorticoid is molded or extruded prior to curing. In one embodiment, the insert or implant has an essentially cylindrical shape, with an essentially round cross-section. The shape of the insert or implant produced from extrusion may also be described as a fiber, strand or rod.

Other geometries of the outer insert or implant shape or its cross-section may also be used such as armed star shape, gear shape, ribbon shape (e.g., flat rectangular), circle or semi-circle shape or trapezoid shape. For example, instead of the round fiber, an oval (or elliptical) fiber may be used. As long as the insert or implant expands in diameter upon hydration in the canaliculus to an average hydrated diameter as disclosed herein, the exact cross-sectional shape is not decisive, as tissue will form around the insert.

The polymer network, such as the PEG network, of the hydrogel insert or implant according to certain embodiments of the present invention may be semi-crystalline in the dry state at or below room temperature, and amorphous in the wet state. Even in the stretched form, the dry insert or implant may be dimensionally stable at or below room temperature, which may be advantageous for administering the insert or implant into the target tissue, and also for quality control.

In certain embodiments, this dimensional change is enabled at least in part by the “shape memory” effect introduced into the insert or implant by means of stretching the insert or implant in the longitudinal direction during its manufacture as also disclosed herein. In certain embodiments, this stretching may be performed in the wet state, i.e., before drying. However, in certain other embodiments, the stretching of the hydrogel strands (once cured) may be performed in the dry state (i.e., after drying the hydrogel strands). It is noted that if no stretching is performed at all the insert or implant may merely swell due to the uptake of water, but the dimensional change of an increase in diameter and a decrease in length disclosed herein may not be achieved, or may not be achieved to a large extent. This could result in a less than optimal fixture of the insert or implant in the canaliculus, for example, and could potentially lead to the insert or implant being cleared (potentially even prior to the release of the complete dose of the active agent) through the nasolacrimal duct or through the punctum. If this is not desired, the hydrogel fiber may e.g. be dry or wet stretched in order to provide for expansion of the diameter upon rehydration.

In the hydrogels of the present invention, a degree of molecular orientation may be imparted by stretching the material then allowing it to solidify, locking in the molecular orientation. The molecular orientation provides one mechanism for anisotropic swelling upon contacting the insert or implant with a hydrating medium such as tear fluid. Upon hydration, the insert or implant of certain embodiments of the present invention will swell only in the radial dimension, while the length will either decrease or be essentially maintained. The term “anisotropic swelling” means swelling preferentially in one direction as opposed to another, as in a cylinder that swells predominantly in diameter, but does not appreciably expand (or does even contract) in the longitudinal dimension.

The degree of dimensional change upon hydration may depend inter alia on the stretch factor. Merely as an example to illustrate the effect of stretching, stretching at e.g. a stretch factor of about 1.3 (e.g. by means of wet stretching) may have a less pronounced effect or may not change the length and/or the diameter during hydration to a large extent. In contrast, stretching at e.g. a stretch factor of about 1.8 (e.g. by means of wet stretching) may result in a shorter length and/or an increased diameter during hydration. Stretching at e.g. a stretch factor of about 3 or 4 (e.g. by means of dry stretching) could result in a much shorter length and a much larger diameter upon hydration. One skilled in the art will appreciate that other factors besides stretching can also affect swelling behavior.

Among other factors influencing the possibility to stretch the hydrogel and to elicit dimensional change of the insert or implant upon hydration is the composition of the polymer network. In the case PEG precursors are used, those with a lower number of arms (such as 4-armed PEG precursors) contribute to providing a higher flexibility in the hydrogel than those with a higher number of arms (such as 8-armed PEG precursors). If a hydrogel contains more of the less flexible components (e.g. a higher amount of PEG precursors containing a larger number of arms, such as the 8-armed PEG units), the hydrogel may be firmer and less easy to stretch without fracturing. On the other hand, a hydrogel containing more flexible components (such as PEG precursors containing a lower number of arms, such as 4-armed PEG units) may be easier to stretch and softer, but also swells more upon hydration. Thus, the behavior and properties of the insert or implant once it has been administered and is rehydrated can be tailored by means of varying structural features as well as by modifying the processing of the insert or implant after it has been initially formed.

The dried insert or implant dimensions inter alia depend on the amount of glucocorticoid incorporated as well as the ratio of glucocorticoid to polymer units and can additionally be controlled by the diameter and shape of the mold or tubing in which the hydrogel is allowed to gel. The diameter of the dried insert or implant may be further controlled by (wet or dry) stretching of the hydrogel strands once formed as disclosed herein. The dried hydrogel strands (after stretching) are cut into segments of the desired length to form the insert; the length can thus be chosen as desired.

### Release of the Active and Biodegradation of the Insert:

In certain embodiments, it has been found that the persistence, i.e., the time to disappearance in vivo or in vitro) of the inserts or implants of the present invention is increased as compared to other methods such as casting. In one embodiment, the present invention relates to a sustained release biodegradable ocular insert or implant comprising a hydrogel and an active agent, wherein the insert or implant provides for a release of a therapeutically effective amount of the active agent for a period from about 1 day to about 14 months or more. In other embodiments, the release is 30 days or less, 2 months or less, 3 months or less, or 6 months or less, or 9 months or less, or 11 months or less, or 12 months or less, or 13 months or less, or 14 months or less, or 15 months or less.

### Further Embodiments

In certain embodiments, the formulation comprises from about 1 to about 80% active ingredient by weight, and from about 20% to about 80% polyethylene glycol by weight.

In certain embodiments, the polyethylene glycol comprises 4a20 k SG, 4a20 k SAP, 4a20 k SAZ, 8a20 k NH3+, TLA crosslinker or a combination thereof.

The active ingredients can be, e.g., dexamethasone, cyclosporine, axitinib, bupivacaine, ropivacaine or other suitable active ingredient used to treat various eye disorder or diseases. In certain embodiments, the batch size of the formulations may be from about 25 g to about 300 g. In other embodiments, the batch size can be up to 1 kg or more.

In certain embodiments, mixing the materials prior to extrusion comprises hand mixing (e.g., in a sealable plastic bag), in a mechanical mixer such as a FlakTek Speed Mixer or a v-shell blender or in a mixer utilizing sound waves such as a Resodyn Acoustic Mixer.

In certain embodiments, the powder feed rate into the extruder is from about 2 g/min to about 10 g/min. the powder feeder can be a plunger feeder or a K-Tron or Brabender feeder.

In certain embodiments, the extrusion processing temperature is from about 20 C to about 150 C or about 40 C to about 90 C. In certain embodiments, the polyethylene glycol is melted and the active ingredient is not melted.

In certain embodiments, the die size of the extruder is from about 0.3 mm to about 3 mm and can utilize different shapes (e.g., cross or star) to optimize surface area.

In certain embodiments, the screw speed of the extruder is from about 50 rpm to about 200 rpm.

### EXAMPLES

The following Examples are included to demonstrate certain aspects and embodiments of

the invention as described in the claims. It should be appreciated by those of skill in the art, however, that the following description is illustrative only and should not be taken in any way as a restriction of the invention.

### Example 1

Extrusion Run Using a MiniCTW Extruder

The melt extrusion process begins with obtaining the necessary raw materials. This includes the reactive polymers (4a20K PEG SG and fluorescein tagged trilysine acetate (TLA)), the API, and Sodium Phosphate Dibasic. Alternatively, the TLA can be substituted with a PEG amine salt. These materials are first combined and mixed for 10 minutes in the melt or powder form to provide a homogenous pelletized, granulated or blended powder material. The material is then loaded into the MiniCTW melt extruder (Thermofisher, Inc.), which has been set to temperature (50-55° C.) and screw rotation speed (20 rpm). The material may be recirculated within the barrel of the twin-screw mixing extruder for 10 minutes to confirm homogeneity before extrusion. Material can then be extruded through the die of the extruder onto a conveyer belt at a speed of 1000 RPM (1.4 in/sec). The rate of drawing determines the diameter of the extrudate. Drawing keeps material straight and allows it to cool and harden before being cut away from the extruder and collected for downstream processing. After extrusion, material can be placed in a humidity chamber to crosslink, typically overnight, for a period of 16-24 hours. After crosslinking, the damp, rubbery material can be stretched to its final length and then dried overnight, at which point it is ready to be cut and inspected in the same manner it would be in a liquid casting process. The process is performed with the exclusion of water during extrusion, which facilitates activation of the PEG crosslinking reaction. The extrusion was run at low temperature, 50-55° C., since heat is not required to drive a crosslinking reaction. Exposure to a controlled water vapor environment (>95% humidity) after extrusion allows enough water to penetrate the strand to activate the curing reaction. The dampened strand, once crosslinked, is a rubber, which can be stretched at 3×. Evaporative drying with nitrogen sweep leaves a semi-crystalline solid with the same molecular and physical structure and properties of dried Dextenza® (Dexamethasone intracanalicular insert) strand, albeit not by casting.

Table 1 outlines these steps, and considers exemplary equipment for each step as well as exemplary settings for each step.

TABLE 1
Laboratory bench top process steps

| Processing Step | Equipment/Material Required | Equipment/Material Specs |
|---|---|---|
| Material weights | 4a20K PEG SG | 50% |
| | TLA | 4% |
| | API | 45% |
| | Sodium phosphate dibasic | 1% |
| | Balance | Analytical balance |
| Material | Hot plate | 100° C. for material melt |
| melting/mixing | Spatulas | Mix until visually uniform |
| | Aluminum weigh boat | |
| Pelletizing/crushing | Stainless Spatula | Crush cooled material until |
| | | average size of~2 mm diameter, |
| | | small enough to fit into the throat |
| | | of the extruder without clogging |
| Material | Funnel | Funnel with coupling attached to |
| Recirculation | Thermo Fisher HAAKE | the throat of the extruder |
| | MiniCTW extruder | Extruder settings: |
| | | 100 RPM mix |
| | | 55° C. barrel temp |
| | | 10 minute mix time |
| Extrusion | Thermo Fisher mini CTW | Extruder settings: |
| | extruder | 20 RPM mix/extrude |
| | | 55° C. barrel temp |
| Conveying/uptake | Mini-mover conveyer | Conveyer settings: |
| | Model/Part #: 20-02036-R-N | 1000 RPM = 1.4 in/sec |
| | U1 RI-050N-20-31 | |
| | Friction applying wheel | |
| Crosslinking | Passive Humidity Chamber | Humidity inside chamber: 100% |
| | Crosslinking Mesh | Crosslink time: 16-24 hrs |
| | RO/DI | |
| | Extech Instruments RHT20 | |
| | Humidity/Temperature | |
| | Data logger | |
| Stretching | ED Stretcher (modular) | Active humidity chamber >95% |
| | Stretching base | humidity |
| | Drill w/coupling | Stretch 3× length (measured from |
| | Active humidity/drying | inside of clamps) |
| | chamber | Stretched slow and consistent |
| | Modified humidifier | Apply water vapor to strands while |
| | Extech Instruments RHT20 | stretching |
| | Humidity/Temperature | |
| | Data logger | |
| Drying | Active humidity/drying | Convert chamber from active |
| | chamber | humidity chamber to drying |
| | Nitrogen | chamber |
| | | Dry at flow of 20 SCFH overnight |
| | | (16-24 hrs) |
| Downstream | Cutting | No changes have been made to |
| Processes | Inspection | these existing processes |
| | Packaging | Future improvements may be |
| | Conditioning | made |
| | Gamma | |

### Product Composition:

Using the procedures above, a dexamethasone composition was prepared with the components of Table 2.

TABLE 2

| | Melt Extrude |
|---|---|
| | Composition |
| Material | (%) |
| 4a20K PEG SG | 50% |
| TLA w/FI | 3%* |
| Dexamethasone | 45% |
| Sodium Phosphate Dibasic | 2% |
| Sodium Phosphate Monobasic | 0% |
| % Solids at Hydrated Equilibrium | 22% |
| % Water at Hydrated Equilibrium | 78% |

*Pending stoichiometric differences between Dextenza and Melt Extrude

### Results:

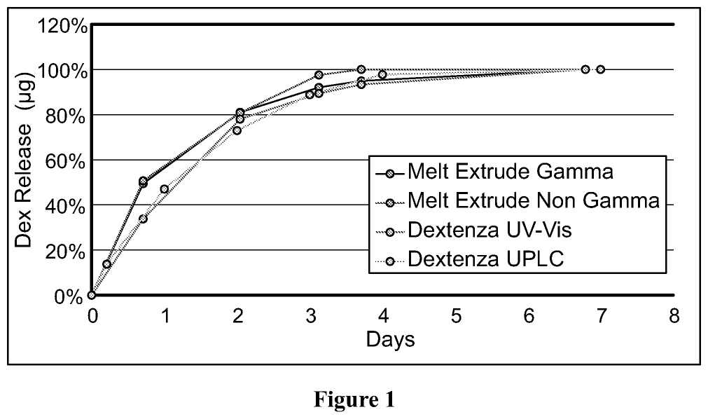

FIG. 1 represents in vitro Release of melt extruded material of the Example compared to Dextenza, as well as pre and post gamma sterilization results.

After processing these batches to completion (including drying and cutting), plugs were analyzed for dry/wet dimensions and compared to Dextenza®. These results can be seen in Table 3. Assay of the melt extruded product was comparable to Dextenza® as shown in Table 4.

TABLE 4

| | | | Length | Diameter | | |
|---|---|---|---|---|---|---|
| | Length | Diameter | 10 min | 10 min | Length | Diameter |
| | Dry | Dry | Hydrated | Hydrated | Hydrated | Hydrated |
| Group | (mm) | (mm) | (mm) | (mm) | (mm) | (mm) |
| Dextenza | 2.85- | <0.55 | N/A | ≥1.00 | N/A | 1.35- |
| Specification | 3.14 | | | | | 1.80 |
| Dextenza Clinical Lot | 2.95 | 0.50 | 2.52 | 1.40 | 2.25 | 1.60 |
| Melt Extrude (A) | 3.07 | 0.52 | 1.53 | 1.55 | 1.62 | 1.58 |
| Melt Extrude (B) | 3.06 | 0.50 | 2.00 | 1.35 | 1.91 | 1.56 |
| Melt Extrude Gamma | 2.98 | 0.51 | 2.17 | 1.25 | 2.04 | 1.47 |
| (ED-415-012) | | | | | | |

TABLE 5

| | Avg. | Avg. | Avg. |
|---|---|---|---|
| | Length | Diameter | Total API |
| Sample | (mm) | (mm) | (μg) |
| DEXTENZA Specification | 2.85-3.14 | <0.55 | 396 μg +/− 39 μg |
| DEXTENZA Clinical Lot | 2.99 | 0.51 | 376 |
| Melt Extrude (A) | 3.03 | 0.52 | 363 |

### Example 2

### Extrusion Run Using Leistritz Nano-16 Extruder

### Formulation: Cyclosporin—75 g Batch Size

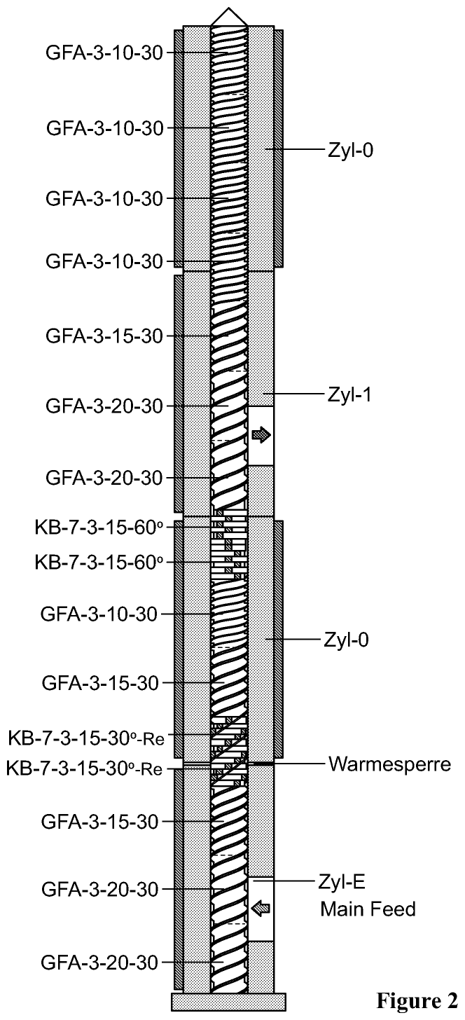

38.1 g of micronized cyclosporine and 32.2 g (50.8%) of 4a20 k PEG SG (42.9%) were placed into a 250 mL bottle and sealed under nitrogen. 0.94 g of trilysine acetate (TLA) and 3.8 g of sodium phosphate dibasic salt were placed into a glass vial and sealed under nitrogen. The materials were mixed and added to the plunger feeder. Air was reduced by tapping with a rubber mallet. Parallel twin screws were set up using the custom configuration in FIG. 2. The two shaded elements were to increase shear, mixing, and degassing of the formulation. The downward arrow in the diagram shows the air vent was open. The plunger temperature was set to “COLD” using the water jacket, set zone #1 to 70° C., zone #2 to 50° C., zone #3 to 40° C. and zone #4 at the die opening to 50° C. The die opening was circular with a diameter of 1.5 mm. The screws were set to 100 rpm with a powder feed rate of 5 cc/min. The Dorner 2200 series conveyor belt collecting the extrudate ran at 4.4 FPM and fed into the Conair CPC 1-12 SD brand combination puller/cutter. Extrudate strand segments were cut to 20 cm and stored in plastic tubing in a nitrogen purged environment and were stored for further processing.

### Example 3

Extrusion Run using Leistritz Nano-16 extruder
Formulation: axitinib—50 g batch size

34.1 g of micronized dexamethasone (surrogate API, 67.8%) and 10.1 g of 4a20 k PEG SAZ (20.0%) were placed into a 250 mL bottle and seal under nitrogen. 5.1 g of 8a20 k NH3+ salt and 1.0 g of sodium phosphate dibasic salt were placed into a glass vial and sealed under nitrogen. The materials were mixed and added to the plunger feeder. Air was reduced by tapping with a rubber mallet. Parallel twin screws were set up using the custom configuration in FIG. 2. Note the two shaded elements were selected to increase shear, mixing, and degassing the formulation. The downward arrow in the diagram shows the air vent was open. The plunger temperature was set to “COLD” using the water jacket, set zone #1 to 70° C., zone #2 to 50° C., zone #3 to 40° C. and zone #4 at the die opening to 50° C. The die opening was circular with a diameter of 1.5 mm. The screws were set to 100 rpm with a powder feed rate of 5 cc/min. The Dorner 2200 series conveyor belt collecting the extrudate ran at 3.8 FPM and fed into the Conair CPC 1-12 SD brand combination puller/cutter. Extrudate strand segments were cut to 20 cm and stored in plastic tubing in a nitrogen purged environment and were stored for further processing.

### Example 4

Extrusion Run using Leistritz Nano-16 extruder
Formulation: dexamethasone—50 g batch size

22.5 g of micronized dexamethasone (44.8%) and 25.0 g of 4a20 k PEG SG (50.0%) were placed into a 250 mL bottle and sealed under nitrogen. 0.7 g of trilysine acetate (TLA) and 1.8 g of sodium phosphate dibasic salt were placed into a glass vial and seal under nitrogen. The materials were mixed and added to the plunger feeder. Air was reduced by tapping with a rubber mallet. Parallel twin screws were set up using the custom configuration in FIG. 2. Note the two shaded elements were selected to increase shear, mixing, and degassing the formulation. The downward arrow in the diagram shows the air vent was open. The plunger temperature was set to “COLD” using the water jacket, set zone #1 to 70° C., zone #2 to 50° C., zone #3 to 40° C. and zone #4 at the die opening to 50° C. The die opening was circular with a diameter of 1.5 mm. The screws were set to 100 rpm with a powder feed rate of 5 cc/min. The Dorner 2200 series conveyor belt collecting the extrudate ran at 3.4 FPM and fed into the Conair CPC 1-12 SD brand combination puller/cutter. Extrudate strand segments were cut to 20 cm and stored in plastic tubing in a nitrogen purged environment and were stored for further processing.

### Example 5

Extrusion Run using Leistritz Nano-16 extruder
Formulation: cyclosporin (SG)—150 g batch size

26.0 g of micronized cyclosporine (52.0%) and 21.0 g of 4a20 k PEG SAP (42.0%) were placed into a 100 g FlakTek cup and sealed under nitrogen. 0.6 g of trilysine acetate (TLA) and 2.4 g of sodium phosphate dibasic salt were placed into a glass vial and sealed under nitrogen. This was repeated 2× for both vessels. The materials were mixed in a FlakTek speed mixer at 1000 rpm for 2×15 s bursts. The mixed material was added to the K-Tron T20 powder feeder. Parallel twin screws were set up using the custom configuration in FIG. 2. Note the two shaded elements were selected to increase shear, mixing, and degassing the formulation. The downward arrow in the diagram shows the air vent was open. The plunger temperature was set to “COLD” using the water jacket, set zone #1 to 40° C., zone #2 to 70° C., zone #3 to 40° C. and zone #4 at the die opening to 50° C. The die opening was circular with a diameter of 1.1 mm. The screws were set to 150 rpm with a powder feed rate of 2-3 g/min. The Dorner 2200 series conveyor belt collecting the extrudate ran at 3.8 FPM and fed into the Conair CPC 1-12 SD brand combination puller/cutter. Extrudate strand segments were cut to 21.6 in. and stored in plastic tubing in a nitrogen purged environment and were stored for further processing.

### Example 6

Extrusion Run using Leistritz Nano-16 extruder
Formulation: axitinib—35 g batch size

23.8 g of micronized axitinib (69.0%) and 9.3 g of 4a20 k PEG SAZ (27.0%) were placed into a 100 g FlakTek cup and sealed under nitrogen. 0.3 g of trilysine acetate (TLA) and 1.1 g of sodium phosphate dibasic salt were placed into a glass vial and sealed under nitrogen. The materials were mixed in a FlakTek speed mixer at 1000 rpm for 2×15 s bursts. The mixed material was added to the plunger feeder. Air was reduced by tapping with a rubber mallet. Parallel twin screws were set up using the custom configuration in FIG. 2. Note the two shaded elements were selected to increase shear, mixing, and degassing the formulation. The downward arrow in the diagram shows the air vent was open. The plunger temperature was set to “COLD” using the water jacket, set zone #1 to 40° C., zone #2 to 70° C., zone #3 to 40° C. and zone #4 at the die opening to 50° C. The die opening was circular with a diameter of 0.7 mm. The screws were set to 150 rpm with a powder feed rate of 5 cc/min. The Dorner 2200 series conveyor belt collecting the extrudate ran at 12 FPM and fed into the Conair CPC 1-12 SD brand combination puller/cutter. Extrudate strand segments were cut to 21.6 in. and stored in plastic tubing in a nitrogen purged environment and were stored for further processing.

### Example 7

Extrusion Run using Leistritz Nano-16 extruder
Formulation: cyclosporin (SAP)—150 g batch size

26.0 g of micronized cyclosporine (52.0%) and 21.0 g of 4a20 k PEG SAP (42.0%) was placed into a 100 g FlakTek cup and sealed under nitrogen. 0.6 g of trilysine acetate (TLA) and 2.4 g of sodium phosphate dibasic salt was added into a glass vial and sealed under nitrogen. This was repeated 2× for both vessels. The materials were mixed in a FlakTek speed mixer at 1000 rpm for 2×15 s bursts. The mixed material was added to the K-Tron T20 powder feeder. Parallel twin screws were set up using the custom configuration in FIG. 2. Note the two shaded elements were selected to increase shear, mixing, and degassing the formulation. The downward arrow in the diagram shows the air vent was open. The plunger temperature was set to “COLD” using the water jacket, set zone #1 to 40° C., zone #2 to 70° C., zone #3 to 40° C. and zone #4 at the die opening to 50° C. The die opening was circular with a diameter of 1.1 mm. The screws were set to 150 rpm with a powder feed rate of 4 g/min. The Dorner 2200 series conveyor belt collecting the extrudate ran at 3.8 FPM and fed into the Conair CPC 1-12 SD brand combination puller/cutter. Extrudate strand segments were cut to 21.6 in. and stored in plastic tubing in a nitrogen purged environment and were stored for further processing.

### Example 8

Extrusion Run using Leistritz Nano-16 extruder
Formulation: bupivacaine (4a40 k SG/TLA)—37.8 g batch size

24.57 g of micronized bupivacaine (65%) and 12.67 g of 4a40 k PEG SG (33.5%) was added into a cup and sealed under nitrogen and mixed on the FlakTek for 30 seconds at 1000 rpm. g of TLA (0.49%) and 0.39 g of sodium phosphate dibasic salt (1.0%) were placed into a glass vial and sealed under nitrogen. The materials were mixed in a FlakTek speed mixer at 1000 rpm for 2×30 second bursts. And added to plunger feeder. Parallel twin screws nwere set up using the custom configuration in FIG. 2. Note the two shaded elements were selected to increase shear, mixing, and degassing the formulation. The downward arrow in the diagram shows the air vent was open. The plunger temperature was set to “COLD” using the water jacket, set zone #1 to zone #2 to 80° C., zone #3 to 60° C. and zone #4 at the die opening to 80° C. The die opening was circular with a diameter of 1.1 mm. The screws were set to 150 rpm with a powder feed rate of 4 g/min. The Dorner 2200 series conveyor belt collecting the extrudate ran at 3.4 FPM and fed into the Conair CPC 1-12 SD brand combination puller/cutter. Extrudate strand segments were cut to 12 in. and stored in plastic tubing in a nitrogen purged environment and were stored for further processing.

### Example 9

Extrusion Run using Leistritz Nano-16 extruder
Formulation: axitinib (4a20 k SAZ/TLA)—35 g batch size

14.13 g of micronized axitinib (39.8%) and 20.07 g of 4a20 k PEG SAZ (57.5%) were placed into a cup and sealed under nitrogen and mixed on the FlakTek for 30 seconds at 1000 rpm.

g of TLA (1.68%) and 0.37 g of sodium phosphate dibasic salt (1.0%) were placed into a glass vial and sealed under nitrogen and mixed in a FlakTek speed mixer at 1000 rpm for 2×30 second bursts. The mixed material is added to the plunger powder feeder. Parallel twin screws are set up using the custom configuration in FIG. 2. Note the two shaded elements were selected to increase shear, mixing, and degassing the formulation. The downward arrow in the diagram shows the air vent was open. The plunger temperature was set to “COLD” using the water jacket, set zone #1 to 40° C., zone #2 to 70° C., zone #3 to 70° C. and zone #4 at the die opening to 50° C. The die opening was circular with a diameter of 0.5 mm. The screws were set to 150 rpm with a powder feed rate of 4 g/min. The Dorner 2200 series conveyor belt collecting the extrudate and fed into the Conair CPC 1-12 SD brand combination puller/cutter. Extrudate strand segments were cut to 12 in. and stored in plastic tubing in a nitrogen purged environment and were stored for further processing.

### Example 10

Extrusion Run using Leistritz Nano-16 extruder
Formulation: dexamethasone (4a20 k SG/TLA)—36.4 g batch size

18.21 g of micronized dexamethasone (50.0%) and 17.35 g of 4a20 k PEG SG (47.6%) were placed into a cup and sealed under nitrogen and mixed on the FlakTek for 30 seconds at 1000 rpm. 0.51 g of TLA (1.39%) and 0.38 g of sodium phosphate dibasic salt (1.0%) were placed into a glass vial and sealed under nitrogen and mixed in a FlakTek speed mixer at 1000 rpm for 2×30 second bursts. The mixed material were added to the plunger powder feeder. Parallel twin screws were set up using the custom configuration in FIG. 2. Note the two shaded elements were selected to increase shear, mixing, and degassing the formulation. The downward arrow in the diagram shows the air vent was open. The plunger temperature was set to “COLD” using the water jacket, set zone #1 to 60° C., zone #2 to 80° C., zone #3 to 60° C. and zone #4 at the die opening to 50° C. The die opening was circular with a diameter of 1.1 mm. The screws were set to 150 rpm with a powder feed rate of 5 cc/min. The Dorner 2200 series conveyor belt collecting the extrudate and fed into the Conair CPC 1-12 SD brand combination puller/cutter. Extrudate strand segments were cut to 12 in. and stored in plastic tubing in a nitrogen purged environment and were stored for further processing.

### Example 11

Extrusion Run using Leistritz Nano-16 extruder
Formulation: dexamethasone (4a20 k SG/TLA) —33.3 g batch size

21.65 g of micronized dexamethasone (65.0%) and 17.35 g of 4a20 k PEG SG (33.0%) were a added into a cup and seal under nitrogen and mixed on the FlakTek for 30 seconds at 1000 rpm. 0.33 g of TLA (0.96%) and 0.35 g of sodium phosphate dibasic salt (1.0%) were placed into a glass vial and sealed under nitrogen and mixed in a FlakTek speed mixer at 1000 rpm for 2×30 second bursts. The mixed material was added to the plunger powder feeder. Parallel twin screws are set up using the custom configuration in FIG. 2. Note the two shaded elements were selected to increase shear, mixing, and degassing the formulation. The downward arrow in the diagram shows the air vent was open. The plunger temperature was set to “COLD” using the water jacket, set zone #1 to 60° C., zone #2 to 80° C., zone #3 to 60° C. and zone #4 at the die opening to 50° C. The die opening was circular with a diameter of 1.1 mm. The screws were set to 150 rpm with a powder feed rate of 5 cc/min. The Dorner 2200 series conveyor belt collecting the extrudate and fed into the Conair CPC 1-12 SD brand combination puller/cutter. Extrudate strand segments were cut to 12 in. and stored in plastic tubing in a nitrogen purged environment and were stored for further processing.

Exemplary Input Ranges
Formulation: API 1-80% by weight, PEG 20-80% by weight
PEGs: 4a20 k SG, 4a20 k SAP, 4a20 k SAZ, 8a20 k NH3+, TLA crosslinker
APIs: dexamethasone, cyclosporine, axitinib (not shown in examples bupivacaine and ropivacaine)
Batch size: 25-300 g using Nano-16
Powder mixing: hand mix in ziplock bag and FlakTek (looking to try v-shell blender and resodyn acoustic mixing as well)
Powder feed rate: 2-10 g/min using the plunger feeder, K-Tron, and Brabender feeders
Screw geometry: one set used for all trials at Leistritz so far, but this could be changed too to add more mixing/degassing segments to the screws
Temperature profile: 40-90 C, goal to melt PEG and not APIs, keep temp low as possible to protect excipient powders and optimize stability
Die size: circle shapes range 0.3-3 mm or other cross/star shapes to optimize surface area Screw speed: 50-200 rpm

Exemplary Equipment Used
Leistritz Nano-16 brand twin screw extruder
Conair CPC 1-12 SD brand combination puller/cutter
Dorner 2200 brand conveyer belt
K-Tron T20 brand powder feeder
Flak Tek Speed mixer

1. A method of preparing a sustained release biodegradable ocular insert or implant comprising extruding a water soluble polymer composition comprising polyethylene glycol and an active pharmaceutical agent to form an insert or implant suitable for ocular administration wherein the method comprises feeding the polymer composition and the active pharmaceutical agent into an extruder; further comprising mixing the components in the extruder; extruding a strand; and cutting the strand into unit dose inserts or implants and wherein the content uniformity of the unit dose insert or implant is within 15%.

2. (canceled)

3. (canceled)

4. The method of claim 1, wherein the polymer composition and the active pharmaceutical agent are fed separately into the extruder.

5. The method of claim 1, wherein the polymer composition and the active pharmaceutical agent are mixed prior to being fed into the extruder wherein the polymer composition and active pharmaceutical agent are melt mixed and milled prior to being fed into the extruder.

6. (canceled)

7. The method of claim 1, further comprising cooling the strand prior to cutting the strand.

8. The method of claim 1, further comprising stretching the strand prior to cutting the strand.

9. The method of claim 8, wherein the stretching is performed under wet conditions, humid conditions, heated conditions, or a combination thereof.

10. The method of claim 8, wherein the stretching is performed under dry conditions, heated conditions, or a combination thereof.

11. The method of claim 1, wherein the extruded composition is subject to a curing step wherein the curing step comprises humidity exposure.

12. (canceled)

13. The method of claim 11, wherein the curing crosslinks the polymer composition.

14. (canceled)

15. The method of claim 1, wherein the extrusion is performed above the melting point of the polymer and the active agent.

16. The method of claim 1, further comprising drying the strand wherein the drying is performed after stretching the strand.

17. (canceled)

18. (canceled)

19. The method of claim 16, wherein the drying comprises desiccation at ambient temperatures.

20. (canceled)

21. (canceled)

22. The method of claim 8, wherein the hydrogel strand is stretched by a stretch factor in the range of about 1 to about 6.

23. (canceled)

24. (canceled)

25. (canceled)

26. (canceled)

27. (canceled)

28. (canceled)

29. The method of claim 1, wherein the content uniformity of the unit dose insert or implant is within 10%.

30. (canceled)

31. (canceled)

32. (canceled)

33. The method of claim 1, wherein the purity of the active agent after curing is greater than 99%.

34. (canceled)

35. (canceled)

36. (canceled)

37. (canceled)

38. (canceled)

39. (canceled)

40. (canceled)

41. The method of claim 1, wherein the polymer composition further comprises a visualization agent.

42. The method of claim 41, wherein the visualization agent is a fluorophore.

43. (canceled)

44. (canceled)

45. The method of claim 1, further comprising forming the extrudate by injection molding.

46. The method of claim 45 comprising injecting the extrudate in a mold cavity and allowing the extrudate to cool and harden into the configuration of the cavity.

47. The method of claim 46, wherein the mold comprises steel.

48. The method of claim 46, wherein the mold comprises aluminum.

49. (canceled)

50. (canceled)

51. (canceled)

52. (canceled)

53. The method of claim 1, wherein the active agent is dexamethasone and the insert or implant provides an in-vitro release of dexamethasone at 1 hour of from about 30% to about 70% wherein the in-vitro release is measured at 37° C. in water with Ultra Performance Liquid Chromatography using an Acquity BEH C8 Column; or by pH4 phosphate buffered saline (PBS) on a Mettler Toledo UV5 Spectrometer.

54. The method of claim 53, wherein the active agent is dexamethasone and the insert or implant provides an in-vitro release of dexamethasone at 2 hours of from about 60% to about 90%.

55. (canceled)

56. (canceled)

57. (canceled)

58. (canceled)

59. (canceled)

60. (canceled)

61. (canceled)
